(19) United States
(12) Patent Application Publication
(10) Pub. No.: US 2025/0032856 A1
(43) Pub. Date: Jan. 30, 2025

(54) Device and Method for Fitness Monitoring with Real-Time Customizable Function Generator

(71) Applicant: CalderBot Technology Ltd., Boulder, CO (US)

(72) Inventor: Calder Daenzer, Boulder, CO (US)

(21) Appl. No.: 18/782,976

(22) Filed: Jul. 24, 2024

Related U.S. Application Data
(60) Provisional application No. 63/529,300, filed on Jul. 27, 2023.

(51) Int. Cl. A63B 24/00; A63B 71/06

(52) U.S. Cl. CPC A63B 24/0075; A63B 71/0622

(57) ABSTRACT

A method of generating a real-time custom fitness parameter with a mobile device having a graphical user interface and the fitness parameter being associated with a fitness activity is disclosed. The method may include creating a user-defined fitness parameter including selecting a first identifier representative of a first variable displayed on the graphical user interface, the first variable based at least in part on a first connected sensor; acquiring numerical data from the first connected sensor representative of a first physical parameter; selecting a second identifier representative of a value displayed on the graphical user interface, the value based on a user inputted constant, a predefined constant, and/or a second variable; and selecting a identifier representative of a mathematical operator displayed on the graphical user interface to operate on the outputted numerical data and the value.

### CROSS REFERENCE TO RELATED APPLICATION

This is an U.S. non-provisional patent application claiming the benefit of and priority to U.S. provisional patent application Ser. No. 63/529,300 filed Jul. 27, 2023, hereby incorporated by reference in its entirety.

### FIELD OF THE INVENTION

The technology disclosed herein relates generally to physical performance data analysis, particularly for cycling activities. Specifically, it relates to a mobile device or smartphone app which may be for use while cycling indoors or outdoors, possibly with the phone mounted on the bicycle handlebars.

### BACKGROUND OF THE INVENTION

Smartphones are capable of collecting and exposing data streams from numerous sensors in real-time (for example accelerometer, gyroscope, altimeter, thermometer, video camera, microphone, GPS, and any external sensors paired via Bluetooth are typically available to a smartphone). This data, when collected during physical activity, can be useful for analyzing an activity both while it is ongoing and after it is complete. As an example, it is desirable for a cyclist to view their speed data from a smartphone GPS while they ride.

However, sensor output may not necessarily be useful in its raw form. For example, viewing the vector output from a smartphone accelerometer and gyroscope in real time may not necessarily be useful for cycling, because road vibrations may cause the output to effectively be indiscernible from noise to the human eye. To get practical useful output, one may need to apply mathematical operations to the sensor data. For example, after applying smoothing functions to accelerometer and gyroscope data, and utilizing a trigonometric formula, it is possible to calculate the slope of the road being ridden (the hill grade), and the grade itself may be a useful metric for cyclists.

For cycling, dedicated bike computers exist as well as apps for handlebar-mounted smartphones that take sensor data and display the outputs of pre-programmed formulas applied to this data. For example, hill grade, speed, cadence, power output, heart rate, etc. are all desirable metrics that can be derived from sensor data and displayed. Such devices may also provide options such as averages and maxima over certain predefined time periods for those metrics.

It may be desirable for users to create their own formulas that produce metrics they believe may be more useful than and not included in the set of pre-programmed functions provided by the manufacturer. A variety of contexts exist in which a user may want to do this. For example, the user may have individual body characteristics that have not been considered, may be experimenting with non-standard techniques within a sport, or may be at the forefront of their sport and pushing certain boundaries of performance. In these cases, it would be useful to have a graphical user interface that allows for the creation of such functions.

Furthermore, it may be desirable for a user who has created their own formula to modify (fine-tune) that formula during brief pauses in physical activity after they have seen how the formula performs in real-world application. To accomplish this it would be useful for formula creation and modification to be easily and quickly accomplished from within the same smartphone app that displays sensor data.

### SUMMARY OF THE INVENTION

The present application includes a variety of aspects, which may be selected in different combinations based upon the particular application or needs to be addressed. Exemplary embodiments of the present invention disclose methods of generating a real-time custom fitness parameter with a mobile device.

### BRIEF DESCRIPTION OF THE DRAWINGS

FIG. 1 is a top perspective view of a mobile device mounted to handlebars of a bicycle according to an exemplary embodiment of the invention.

FIG. 2 is a representative main dashboard display that involves processes and screen selections for utilizing metrics that can be selected by the user in creating custom metrics.

FIG. 3 is a representative gauge edit display that involves processes and screen selections for utilizing metrics that can be selected by the user in creating custom metrics.

FIG. 4 is a representative profile and variables display that involves processes and screen selections for utilizing metrics that can be selected by the user in creating custom metrics.

FIG. 5 is a representative first formula development display that involves processes and screen selections for utilizing metrics that can be selected by the user in creating custom metrics.

FIG. 6 is a representative operator display that involves processes and screen selections for utilizing metrics that can be selected by the user in creating custom metrics.

FIG. 7 is a representative second formula development display that involves processes and screen selections for utilizing metrics that can be selected by the user in creating custom metrics.

### DETAILED DESCRIPTION

It should be understood that embodiments include a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

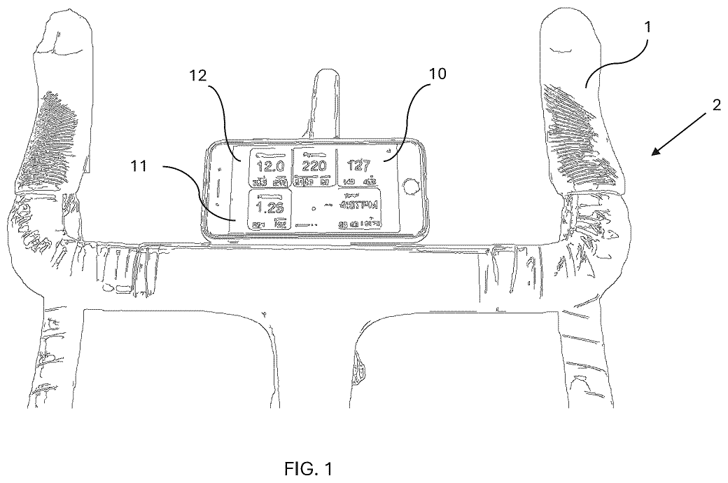

FIG. 1 shows a mobile device 10 detachably secured to handlebars 1 of a bicycle 2, displaying real-time sensor data during a fitness activity, such as bicycling. In some embodiments, the mobile device 10 may be a mobile phone, such as a smartphone, a smartwatch, smart glasses, a tablet, a computer, a stand-alone device, or any combination thereof, and may be mounted to a user or other accessory equipment that is configured to move with the user. For example, the mobile device may be detachably secured to a helmet, boat, piece of equipment, oar, or barbell, dumbbell, secured within a pocket of the user, or attached to an armband of the user. The mobile device 10 may be used in fitness activities characterized by an individual performance, such as running, hiking, climbing, skiing, weightlifting, and rowing or the like with a single participant among other activities. Also, the mobile device 10 may be used in fitness activities characterized by collective individual performances such as rowing with more than one participant. As shown in FIG. 1, the mobile device 10 may include a display 11, on which a mobile app having a graphical user interface (GUI) 12 may be displayed. In other embodiments not shown, the mobile device may communicate with another display, such as video display glasses or a wrist-worn display, for example. In addition, the app may be any form of software on any platform perhaps including a network or website.

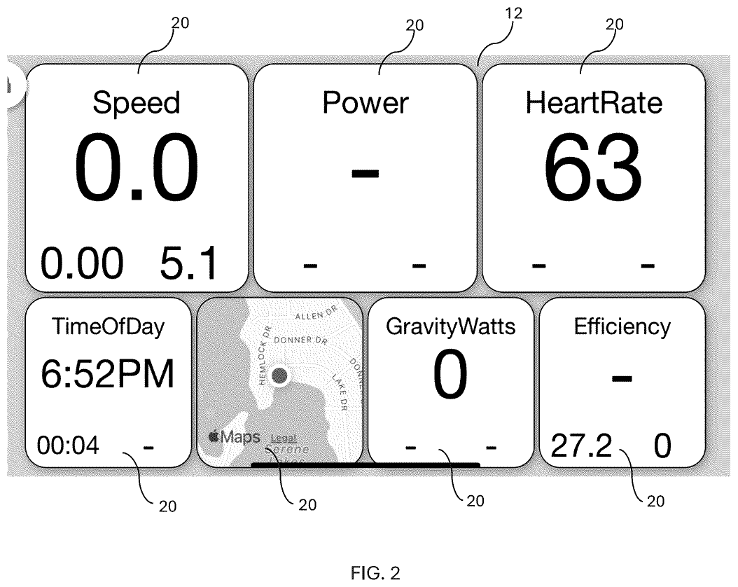

FIG. 2 shows a main dashboard of the GUI 12, where sensor data can be displayed on gauge-displays 20. The gauge-displays 20 may show a visual representation of a value that may include a singular data point, plurality of data, or stream of data including sensor data, variables, maps, or user-defined formulas. As used herein, the term “value” may encompass a singular data point, plurality of data, or stream of data including sensor data, variables, maps, or outputs of user-defined formulas. Exemplary values may include speed, power, heart rate, time of day, moving time, maps (location and route), and user-defined formulas, such as Gravity Watts, and Efficiency. Other user-defined formulas may include SpeedWatts, Gravity Watts, Hill Grade, and VerticalSpeed, for example. These user-defined formulas are discussed in more detail below.

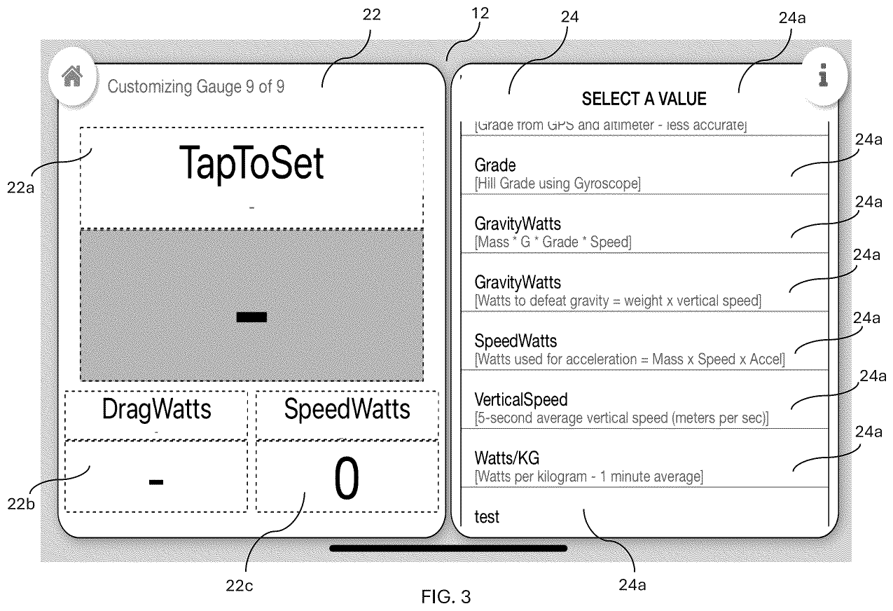

FIG. 3 shows a gauge-edit screen 22, where the gauge-display 20 can be edited or customized. For example, a user may select a “TapToSet” input 22a to select a value, which may bring up a “SELECT A VALUE” input screen 24. Referring back to the gauge-edit screen 22, a user may select miniature displays 24b and 24c to also input values from the “SELECT A VALUE” input screen 24. Referring now to the “SELECT A VALUE” input screen 24, a user may select an input value 24a such as a preloaded value, an uploaded value from community members, or a user-defined value.

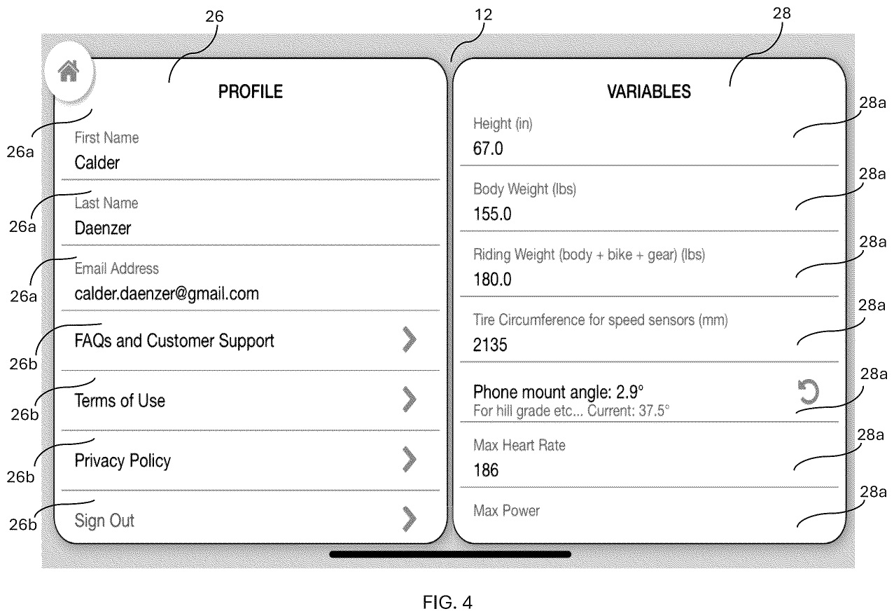

Instead of selecting an input value 24a, a user may create a user-defined fitness parameter. FIG. 4 shows a “PROFILE” input screen 26 where a user may start to create the user-defined fitness parameter. In an exemplary embodiment, the user starts with the “Profile” input screen 26, but in other embodiments, a user may complete this screen after creating the user-defined fitness parameter or may omit this screen altogether. The “PROFILE” input screen 26 may allow a user to input profile data 26a such as background or ancillary data associated with the user, including, for example, the user's name and email address. The “PROFILE” input screen 26 may also allow the user to select background menu items 26b, such as FAQs and Customer Support, Terms of User, Privacy Policy, and Sign Out, for example. FIG. 4 also shows a “VARIABLE” input screen 28 where a user may input user-specific variables 28a, such as height, body weight, riding weight (body weight+bike weight+gear), tire circumference, mobile device mount angle, max heart rate, and max power, for example. The GUI 12 may be configured to populate some of the user-specific variables 28a with previously stored, measured, or calculated values, such as max heart rate and max power, for example.

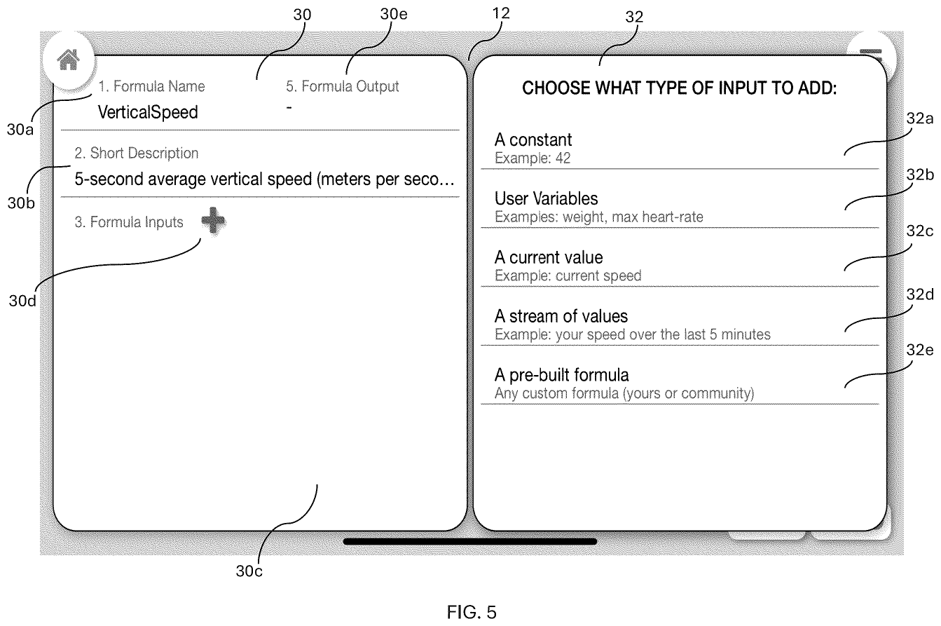

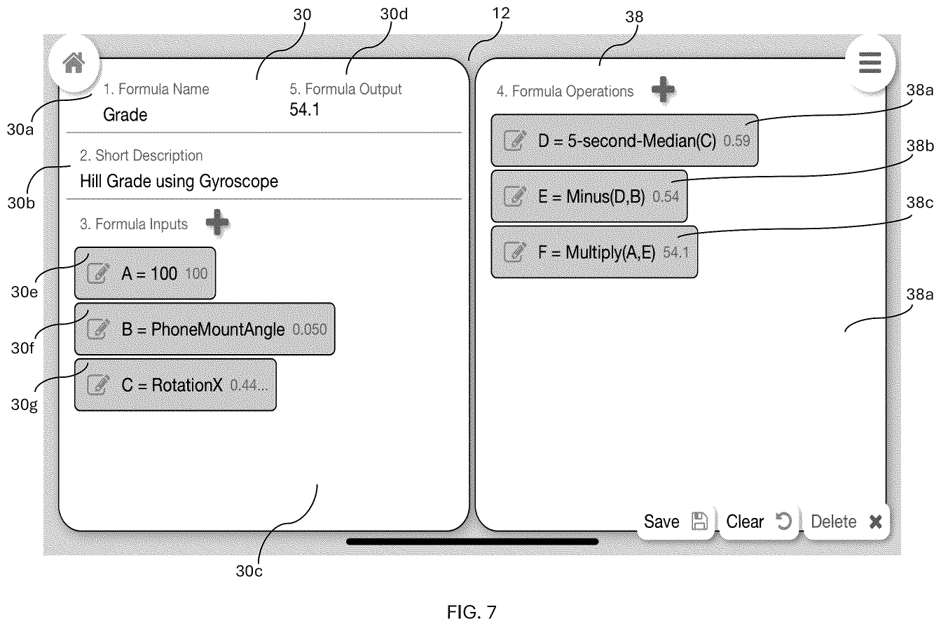

FIGS. 5 and 7 show a formula development screen 30 for creating a user-defined fitness parameter. A user may provide a name for the new fitness parameter in a formula name field 30a and a description in a short description field 30b. The formula development screen 30 also includes a formula input field 30c where a user may select inputs by clicking on a formula input plus sign 30d. Clicking on the formula input plus sign 30d may open an input adding screen 32, where a user may select an identifier 32a-32e representative of an input displayed on the graphical user interface 12, the input may be based on a user inputted constant, a predefined constant, and/or a variable. For example, the identifier 32a-32e may be a constant 32a, a user defined variable 32b (such as weight), a current value 32c (such as current speed), a stream of values 32d (such as speed over the last five seconds), or a pre-built formula 32e (such as one-created by the user or shared by a community of users), among others. In some embodiments, inputting and selecting items on the GUI can be performed by a touchscreen, keyboard, mouse, and/or verbal commands, among other input methods.

Some of the identifiers 32a-32e may be based on a connected sensor. For example, sensors may include an accelerometer, a gyroscope, an altimeter, a thermometer, a video camera, a microphone, a GPS sensor, or any external sensors paired via wired or wireless connection, such as Bluetooth for example. The mobile device 10 may be configured to acquire numerical data from the connected sensor representative of a first physical parameter. A non-exhaustive list for physical parameters include speed, position, temperature, angle, force, etc. The mobile device 10 may acquire analog or digital numerical data whether raw data or conditioned data. The mobile device 10 may be configured to start acquiring data from all or select identified sensors upon opening the mobile app so that the user can see the effects of modifying the formulas in real-time. In some embodiments, the mobile device 10 may acquire data from internal sensors, for example, that are running in the background of the mobile device 10 whether the mobile app has been opened. In addition, the mobile device may be configured to capture numerical data when the mobile app is selected. Capturing data may include collecting and storing or displaying data. Storing data may include storing data in either transient or permanent memory.

In some embodiments, a user may select a first identifier representative of a first input displayed on the graphical user interface, the first input based at least in part on a first connected sensor and select a second identifier representative of a second input displayed on the graphical user interface, the second input based on a user inputted constant, a predefined constant, and/or a second variable. Indeed, the user may repeat this selecting process selecting more than two identifiers. For example, a user may select, with the mobile device, an additional identifier representative of an additional variable displayed on the graphical user interface, the additional variable based on a second connected sensor. The user may select the first, second, or any identifier while the mobile device is acquiring the mobile data. Moreover, a user may select the first, second, or any identifier while the mobile device is capturing the mobile data. Further, the user may perform the fitness activity while selecting any of the identifiers or, in other words, while creating the user-defined fitness parameter.

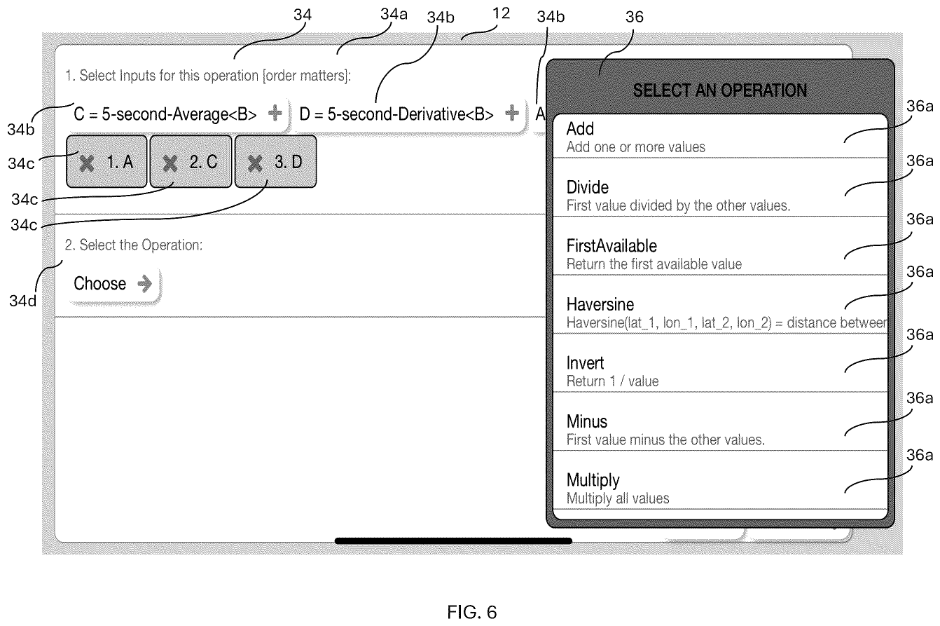

FIG. 6 shows an operation screen 34 that a user may access for selecting a mathematical operator to operate on the previously selected identifiers. The operation screen 34 may include an operation input field 34a showing the previously selected identifiers 32a-32e in text 3b and perhaps in smaller abbreviated identifiers 34c. The operation screen 34 also includes an operation selection field 34d, where a user may select a third identifier representative of a mathematical operator displayed on the graphical user interface to operate on the numerical data and another input, such as the first and/or second input. By clicking on the operation selection field 34d, a “SELECT AN OPERATION” window 36 may open listing various mathematical operations 36a that a user may select. The mathematical operations may include, for example, an arithmetic operator, a trigonometric operator, a statistical operator, a calculus operator, and/or a smoothing operator or the like.

In some embodiments, such as those discussed above, the identifiers are selected in a sequence starting with the first identifier, then the second identifier, and then the last identifier. However, it should be understood that the identifiers may be selected in any sequence and acquiring numerical data may occur at any time before, during, or after selection of any of the identifiers.

The GUI 12 may be configured to allow the user to easily select inputs and operators without having to write script-based code. For example, any of the identifiers selected, such as the first identifier, second identifier, and third identifier may be configured to be automatically accepted upon performing a single touch of the display. In addition, the size of the identifiers lends itself well to easily selecting them. For example, surface area of any of the identifiers, such as the first, second, or third identifier on the graphical user interface may be at least 1% of the total screen size of the display. Also, the surface area of any of the identifiers, such as the first, second, or third identifier on the graphical user interface may be at least 2%, 3%, or 4% of the total screen size of the display.

As shown in FIG. 7, the selected identifiers 32a-32e are shown as individualized boxes 30e, with short-hand temporary variables A, B, C, for example, assigned to each one with a description and value displayed. In addition, the selected operations 36a are shown in a formula operations screen 38 as individualized boxes 38a, with short-hand temporary variables D, E, F, for example, assigned to each one with a description and the short-hand temporary variable on which it operates. The short-hand temporary variables permit a user to simplify operations by, for example, adding A to B and then multiplying by C.

The selected identifiers 32a-32e and 36a may be arranged in the order they are selected according to any user-defined sequence to form a customized relationship therebetween or they may be manually arranged by the user to also form a customized relationship therebetween.

The mobile device 10 may process the selected inputs, such as the first and second inputs, using the selected mathematical operator(s) to create the user-defined fitness parameter. The user-defined fitness parameter may then be displayed as a gauge-display 20 on the main dashboard of the GUI 12 (FIG. 2). In addition, the user may save the user-defined fitness parameter with a unique identifier, such as that entered into the formula name 30a and share it on another mobile device via device-to-device transfer or through a network. In doing so, the user-defined fitness parameter may be uploaded into a library of fitness parameters.

As described above with respect to FIG. 5, a user may select a previously built formula by selecting a corresponding identifier. The user may also create a formula with a customized relationship that is not previously shown on the graphical user interface or stored on the device prior to creating the user defined fitness parameter.

Some exemplary formulas may include the following:

A formula having an output of hill grade. This may be achieved by taking the tangent of the 5-second median of the internal gyroscope rotation angle in the direction of forward motion. There may be a correction term for the phone mount angle (this correction term may be a user-defined variable). Note: A 5-second median may be used instead of the instantaneous angle because rotation is measured every 200 ms but may be quite noisy when cycling, and after testing various smoothing and de-noising operations the 5-second median could provide a good balance of accuracy and timeliness.

A formula having an output of vertical speed. The formula may be a 5-second average speed multiplied by sine (angle), where angle may be the same as discussed above.

A formula having an output of the power used to overcome gravity while cycling on a hill. The formula may be total rider weight (a user-defined variable) multiplied by Newton's constant (a numeric constant) multiplied by vertical speed (a previously defined formula).

A formula having an output of the power used to accelerate the bicycle. The formula may be total rider weight multiplied by a 5-second average speed multiplied by a 5-second discrete derivative of speed. (The last component acts as the average acceleration over 5 seconds.)

A formula having an output of the power used to overcome all frictional forces. Since the total power output of a cyclist is the sum of power to accelerate, power to overcome gravity, and power to overcome frictional forces, the formula for this may be a 5-second average power output minus the power to overcome gravity minus the power to accelerate the bicycle.

A formula having an output of physical efficiency. The formula may be a 30-second average power divided by heart rate. Heart rate is a lagging indicator of physical activity (lagging by roughly 30 seconds), so this formula may use the 30-second average power but the current heart rate value. This formula may be most useful when a user experiments with different cycling techniques, different clothing, body cooling techniques, amount of rest, and other factors to try to maximize this value.

As can be easily understood from the foregoing, the basic concepts of the various embodiments of the present invention(s) may be embodied in a variety of ways. It involves both fitness monitoring techniques as well as devices to accomplish the fitness monitoring. In this application, the fitness monitoring techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “selection” should be understood to encompass disclosure of the act of “selecting”—whether explicitly discussed or not—and, conversely, were there effectively disclosure of the act of “selecting,” such a disclosure should be understood to encompass disclosure of a “selection” and even a “means for selecting.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components, programming, subroutines, logic, or elements that are configured to, or configured and arranged to, provide or even achieve a particular result, use, purpose, situation, function, or operation, or as components that are capable of achieving a particular activity, result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the listing below or other information statement filed with the application are hereby appended and hereby incorporated by reference, however, as to each of the above, to the extent that such information or statements incorporated by reference might be considered inconsistent with the patenting of the various embodiments of invention(s) such statements are expressly not to be considered as made by the applicant(s). The attached Appendix is hereby incorporated by reference into this patent application.

Finally, all references listed in the list of References To Be Incorporated By Reference or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make claims to embodiments including at least: i) each of the fitness monitoring devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the various embodiments of the invention(s)—whether characterized as a device, a capability, an element, or otherwise, because all of these can be implemented via software, hardware, or even firmware structures as set up for a general purpose computer, a programmed chip or chipset, an ASIC, application specific controller, subroutine, logic, or other known programmable or circuit specific structure—it should be understood that all such aspects are at least defined by structures including, as person of ordinary skill in the art would well recognize: hardware circuitry, firmware, programmed application specific components, and even a general purpose computer programmed to accomplish the identified aspect. For such items implemented by programmable features, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines, processor logic, and/or programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, and the like) that act to sequence and/or cause actions as detailed, xxvii) computer readable medium(s) storing instructions to execute the steps and cause activities detailed, xxviii) the related methods disclosed and described, xxix) similar, equivalent, and even implicit variations of each of these systems and methods, xxx) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

In addition, the applicant(s) should be understood to have support to claim and make a statement of invention that may include claims directed to:
Creating and providing displays in real-time of advanced and perhaps complex metrics for cycling and other applications;
Providing a system to facilitate users and others to alter metrics and even create custom metrics for cycling and other applications;
Systems and processes for simplified creation of customized metrics in an athletic endeavor;
Providing a system to allow users to share custom metrics such as for cycling and other applications;
Establishing a hardware, data, and software/firmware system for improved cycling performance;
Configuring existing computational capabilities for use as specialized cycling monitors and cycling aids;
Displaying in real time cycling performance metrics including even uniquely created personal metrics;
Customizing displays such as for real time cycling performance monitoring;
Collecting, making available, and utilizing multiple users' data to facilitate and allow machine learning and other advanced data analysis and generation capabilities among a collective of users and contributors;
Determining different and even unique measures of performance in real time and afterwards for activities such as for cycling and other applications; and
Any permutation or combination of any of the above or any of the elements of the above and as otherwise described in this disclosure.

Clauses for potential assertion include:

A method of generating a real-time custom fitness parameter with a mobile device having a graphical user interface and the fitness parameter being associated with a fitness activity, the method comprising:
creating a user-defined fitness parameter comprising:
   selecting, with the mobile device, a first identifier representative of a first input displayed on the graphical user interface, the first input based at least in part on a first connected sensor;
   acquiring, with the mobile device, numerical data from the first connected sensor representative of a first physical parameter,
   selecting, with the mobile device, a second identifier representative of a second input displayed on the graphical user interface, the second input based on a user inputted constant, a predefined constant, and/or a second variable;
   selecting, with the mobile device, a third identifier representative of a mathematical operator displayed on the graphical user interface to operate on the numerical data and the first input; and
   processing, with the mobile device, the first input and the second input using the mathematical operator to create the user-defined fitness parameter; and
displaying, with a display, the user-defined fitness parameter.

The method as described in any clause, wherein selecting the first identifier is performed while acquiring the numerical data.

The method as described in any clause, wherein acquiring numerical data comprises capturing numerical data and wherein selecting the first identifier is performed while capturing the numerical data.

The method as described in any clause, wherein the fitness activity is characterized by an individualized performance.

The method as described in any clause, wherein the fitness activity is characterized by collective individualized performances.

The method as described in any clause, wherein the fitness activity is one of bicycling, running, hiking, climbing, skiing, weightlifting, and rowing or the like.

The method as described in any clause, wherein the mobile device is detachably secured to the user or accessory equipment while performing the fitness activity, the accessory equipment configured to move with the user.

The method as described in any clause, wherein the fitness activity is bicycling and the mobile device is mounted to handlebars of a bicycle while performing the bicycling.

The method as described in any clause, wherein creating a user-defined fitness parameter further comprises arranging, with the mobile device, at least the first identifier, the second identifier, and the third identifier to form a user customized relationship therebetween.

The method as described in any clause, whereby arranging is performed by selecting the first identifier, the second identifier, and the third identifier in any user-defined sequence.

The method as described in any clause, wherein another identifier representing the user customized relationship is not previously shown on the graphical user interface prior to creating the user defined fitness parameter.

The method as described in any clause, wherein another identifier representing the user customized relationship is not previously stored on the device prior to creating the user defined fitness parameter.

The method as described in any clause, further comprising performing the fitness activity while creating the user-defined fitness parameter.

The method as described in any clause, wherein the mathematical operator includes an arithmetic operator, trigonometric operator, statistical operator, calculus operator, and/or smoothing operator or the like.

The method as described in any clause, further comprising:
selecting, with the mobile device, a fourth identifier representative of a third variable displayed on the graphical user interface, the third variable based at least in part on a second connected sensor; and
acquiring, with the mobile device, second numerical data from the second connected sensor representative of a second physical parameter.

The method as described in any clause, further comprising saving the user-defined fitness parameter with a unique identifier and uploading the user-defined fitness parameter into a library of fitness parameters.

The method as described in any clause, further comprising saving the user-defined fitness parameter with a unique identifier and sharing the user-defined fitness parameter onto another mobile device.

The method as described in any clause, wherein the display is on the mobile device.

The method as described in any clause, wherein selecting any of the first identifier, second identifier, and third identifier is configured to be automatically accepted.

The method as described in any clause, wherein selecting any of the first identifier, second identifier, and third identifier is performed by a single touch on the display.

The method as described in any clause, wherein the surface area of first identifier on the graphical user interface is at least 1% of the total screen size of the display.

The method as described in any clause, wherein the surface area of first identifier on the graphical user interface is at least 2% of the total screen size of the display.

The method as described in any clause, wherein the surface area of first identifier on the graphical user interface is at least 3% of the total screen size of the display.

The method as described in any clause, wherein the surface area of second identifier on the graphical user interface is at least 1% of the total screen size of the display.

The method as described in any clause, wherein the surface area of second identifier on the graphical user interface is at least 2% of the total screen size of the display.

The method as described in any clause, wherein the surface area of second identifier on the graphical user interface is at least 3% of the total screen size of the display.

The method as described in any clause, wherein the surface area of third identifier on the graphical user interface is at least 1% of the total screen size of the display.

The method as described in any clause, wherein the surface area of third identifier on the graphical user interface is at least 2% of the total screen size of the display.

The method as described in any clause, wherein the surface area of third identifier on the graphical user interface is at least 3% of the total screen size of the display.

The method as described in any clause, wherein selecting the first identifier, selecting the second identifier, selecting the third identifier, and acquiring the numerical data, are performed in any sequence.

The method as described in any clause, wherein the display is on another mobile device.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123 (2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrases “comprising,” “including,” “containing,” “characterized by,” and “having” are used to maintain the “open-end” claims herein, according to traditional claim interpretation including that discussed in MPEP § 2111.03. Thus, unless the context requires otherwise, it should be understood that the terms “comprise” or variations such as “comprises” or “comprising,” “include” or variations such as “includes” or “including,” “contain” or variations such as “contains” and “containing,” “characterized by” or variations such as “characterizing by,” “have” or variations such as “has” or “having,” are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method of generating a real-time custom fitness parameter with a mobile device having a graphical user interface and the fitness parameter being associated with a fitness activity, the method comprising:
creating a user-defined fitness parameter comprising:
selecting, with the mobile device, a first identifier representative of a first input displayed on the graphical user interface, the first input based at least in part on a first connected sensor;
acquiring, with the mobile device, numerical data from the first connected sensor representative of a first physical parameter;
selecting, with the mobile device, a second identifier representative of a second input displayed on the graphical user interface, the second input based on a user inputted constant, a predefined constant, and/or a second variable;
selecting, with the mobile device, a third identifier representative of a first mathematical operator displayed on the graphical user interface to operate on the numerical data and the first input; and
processing, with the mobile device, the first input and the second input using the first mathematical operator to create the user-defined fitness parameter; and
displaying, with a display, the user-defined fitness parameter;
wherein selecting the first identifier, second identifier, and third identifier is performed while acquiring and storing the numerical data;
wherein the fitness activity is bicycling and the mobile device is detachably mounted to handlebars of a bicycle while performing the bicycling;
wherein another identifier representing the user customized relationship is not previously shown on the graphical user interface prior to creating the user defined fitness parameter;
wherein selecting the first identifier, second identifier, and third identifier are large enough to be tapped with a finger and configured to be automatically accepted by a single touch on the display; and
wherein selecting the first identifier, selecting the second identifier, selecting the third identifier, and acquiring and storing the numerical data, are performed in any sequence.

2. The method as described in claim 1, wherein the first mathematical operator includes an arithmetic operator, trigonometric operator, statistical operator, calculus operator, and/or smoothing operator.

3. The method as described in claim 1, further comprising saving the user-defined fitness parameter with a unique identifier and sharing the user-defined fitness parameter onto another mobile device.

4. The method as described in claim 1, wherein the display is on another mobile device.

5. The method as described in claim 1, wherein another identifier representing the user customized relationship is not previously stored on the device prior to creating the user defined fitness parameter.

6. The method as described in claim 1, wherein creating a user-defined fitness parameter further comprises arranging, with the mobile device, at least the first identifier, the second identifier, and the third identifier to form a user customized relationship therebetween.

7. The method as described in claim 6, whereby arranging is performed by selecting the first identifier, the second identifier, and the third identifier in any user-defined sequence.

8. The method as described in claim 1, wherein creating a user-defined fitness parameter creates a first user-defined fitness parameter, and the method further comprises:
selecting, with the mobile device, a fourth identifier representative of a third input displayed on the graphical user interface, the third input based on a user inputted constant, a predefined constant, and/or a third variable; and
selecting, with the mobile device, a fifth identifier representative of a second mathematical operator displayed on the graphical user interface; and
processing, with the mobile device, the first user-defined fitness parameter and the third input using the second mathematical operator to create a second user-defined fitness parameter;
wherein displaying, with a display, the user-defined fitness parameter comprises displaying the second user-defined fitness parameter.

9. The method as described in claim 1, further comprising performing the fitness activity while creating the user-defined fitness parameter.

10. The method as described in claim 1, selecting any of the first identifier, second identifier, and third identifier is configured to be automatically accepted by a single touch on the display.

11. The method as described in claim 1, wherein the surface area of first identifier on the graphical user interface is at least 1% of the total screen size of the display.

12. A method of generating a real-time custom fitness parameter with a mobile device having a graphical user interface and the fitness parameter being associated with a fitness activity, the method comprising:
creating a user-defined fitness parameter comprising:
selecting, with the mobile device, a first identifier representative of a first input displayed on the graphical user interface, the first input based at least in part on a first connected sensor;
acquiring, with the mobile device, numerical data from the first connected sensor representative of a first physical parameter;
selecting, with the mobile device, a second identifier representative of a mathematical operator displayed on the graphical user interface to operate on the numerical data and the first input; and
processing, with the mobile device, the first input using the mathematical operator to create the user-defined fitness parameter; and
displaying, with a display, the user-defined fitness parameter;
wherein selecting the first identifier is performed while acquiring the numerical data;
wherein the fitness activity is characterized by an individualized performance and the mobile device is detachably secured to the user or accessory equipment while performing the fitness activity, the accessory equipment configured to move with the user; and
wherein selecting the first identifier, selecting the second identifier, and capturing the numerical data, are performed in any sequence.

13. The method as described in claim 12, wherein the fitness activity is one of bicycling, running, hiking, climbing, skiing, weightlifting, and rowing.

14. The method as described in claim 12, wherein the fitness activity is bicycling and the mobile device is mounted to handlebars of a bicycle while performing the bicycling.

15. The method as described in claim 12, wherein the second identifier is a previously created user-defined fitness parameter.

16. The method as described in claim 12, wherein creating a user-defined fitness parameter creates a first user-defined fitness parameter, and the method further comprises creating a second user-defined fitness parameter, wherein creating the second user-defined fitness parameter comprises:
selecting, with the mobile device, a third identifier representative of a third input displayed on the graphical user interface, the third input based at least in part on a on a user inputted constant, a predefined constant, and/or a second variable;
selecting, with the mobile device, a fourth identifier representative of another mathematical operator displayed on the graphical user interface to operate on first user-defined fitness parameter and the third input, and
processing, with the mobile device, the third input and the first user-defined fitness parameter using the another mathematical operator to create the second user-defined fitness parameter; and
displaying, with a display, the second user-defined fitness parameter.

17. The method as described in claim 12, another identifier representing the user customized relationship is not previously shown on the graphical user interface prior to creating the user defined fitness parameter.

18. The method as described in claim 12, wherein selecting either the first identifier or the second identifier is configured to be automatically accepted by a single touch on the display.

19. The method as described in claim 12, wherein the surface area of first identifier on the graphical user interface is at least 1% of the total screen size of the display.

20. A method of generating a real-time custom fitness parameter with a mobile device having a graphical user interface and the fitness parameter being associated with a fitness activity, the method comprising:
creating a user-defined fitness parameter comprising:
selecting, with the mobile device, a first identifier representative of a first input displayed on the graphical user interface, the first input based at least in part on a first connected sensor;
acquiring, with the mobile device, numerical data from the first connected sensor representative of a first physical parameter;
selecting, with the mobile device, a second identifier representative of a second input displayed on the graphical user interface, the second input based on a user inputted constant, a predefined constant, and/or a second variable;
selecting, with the mobile device, a third identifier representative of a mathematical operator displayed on the graphical user interface to operate on the numerical data and the first input; and
processing, with the mobile device, the first input and the second input using the mathematical operator to create the user-defined fitness parameter; and
displaying, with a display, the user-defined fitness parameter;
wherein creating a user-defined fitness parameter further comprises arranging, with the mobile device, at least the first identifier, the second identifier, and the third identifier to form a user customized relationship therebetween;
wherein another identifier representing the user customized relationship is not previously shown on the graphical user interface prior to creating the user defined fitness parameter,
wherein selecting any of the first identifier, second identifier, and third identifier is configured to be automatically accepted by a single touch on the display; and
wherein selecting the first identifier, selecting the second identifier, selecting the third identifier, and capturing the numerical data, are performed in any sequence.
